(19) United States
(12) Patent Application Publication
(10) Pub. No.: US 2015/0164590 A1
(43) Pub. Date: Jun. 18, 2015

(54) CONTAINER FOR WASTE SYRINGE NEEDLE

(71) Applicant: IWATSUKI CO., LTD., Tokyo (JP)

(72) Inventors: Shuichi SAKIHAMA, Tokyo (JP); Michiko KATO, Tokyo (JP)

(73) Assignee: IWATSUKI CO., LTD., Tokyo (JP)

(21) Appl. No.: 14/105,894

(22) Filed: Dec. 13, 2013

(51) Int. Cl. A61B 19/02; A61M 5/32

(52) U.S. Cl. CPC A61B 19/0262; A61M 5/3205; A61M 2005/3206

(57) ABSTRACT

A container separates a needle unit from a pen syringe and houses the needle unit. An insertion opening that receives a needle unit has an inner diameter slightly smaller than the outer diameter of the needle unit. When a user inserts the needle unit of a used pen syringe into the insertion opening and rotates the syringe against the needle unit fixed by the linear protrusions inside the insertion opening engaging with the linear protrusions around the needle unit in the rotational direction, the syringe is unscrewed from the needle unit. When the closer is closed while the separated needle unit remains in the insertion opening, a pusher provided on the lower surface of the closer pushes the needle unit downward and drops the needle unit into the container body.

### TECHNICAL FIELD

The present invention relates to a container for waste syringe needles that separates used needle units from syringes and safely houses the separated needle units.

### BACKGROUND ART

In general, a needle unit including a syringe needle used for a medical treatment such as blood collection or drip infusion is contaminated with blood or body fluid of a patient. An accidental contact of a person to the needle unit may cause a puncture wound that leads to infectious diseases or other troubles. The used needle units must therefore be carefully handled as medical waste.

If the whole used syringes are housed in a container as they are, however, the container will be filled with syringes in a short time. In addition, the medical wastes including used needle units contain sharp components and therefore require especially high disposal costs. This leads to a demand to separate needle units from syringes and dispose of the needle units alone.

As is disclosed in, for example, Registered Japanese Design Registration No. 1263856 (Patent Literature 1), Registered Japanese Design Registration No. 1321996 (Patent Literature 2), and Japanese Unexamined Patent Application Publication No. 2009-219833 (Patent Literature 3), conventional techniques separate used needle units from syringes, put the needle units alone into a container for waste syringe needles, and discard the container as a medical waste containing sharp components.

Patent Literatures 1 and 2, which are applicant's Japanese design registrations disclose a lid of a container for waste syringe needles, which can accept needle units used for blood collection and butterfly needles used for drip infusion in a medical treatment.

Various shapes of medical syringes are proposed depending on the usage. Widely used syringes include syringes for general purposes such as blood collection, syringes with butterfly needles for drip infusion, and pen syringes for self-injection of insulin or growth hormone.

### SUMMARY OF INVENTION

### Technical Problem

A variety of conventional containers for waste syringe needles are applicable to syringes for general purposes such as blood collection, as disclosed in Patent Literatures 1 and 2. In contrast, there are a few containers for waste syringe needles applicable to pen syringes. Patent Literature 3 discloses a container for medical wastes applicable to pen syringes, but fails to take safety measures.

The container disclosed in Patent Literature 3 allows a needle case to be attached to the needle unit of a used pen syringe and then the needle unit capped with the needle case to be separated. The attachment of the needle case to the used needle unit, however, requires risky troublesome operations. Although the tip of the needle is covered with the needle case, the syringe-side end of the needle is exposed. A user still needs to bring the needle unit into an opening of the container by risky handwork.

Japanese Unexamined Patent Application Publication No. 2007-29747 (Patent Literature 4) discloses a container for waste syringe needles that retains syringe-needle holders in an insertion opening. According to Patent Literature 4, the syringe-needle holders retained in the insertion opening are dropped by any means into the container upon the disposal of a subsequent used syringe needle.

The syringe-needle holder including the needle with an exposed end will remain on the top of the container until the subsequent used needle is disposed. This may lead to a punctual wound due to an accident such as a careless finger contact.

An object of the present invention, which has been accomplished to solve the above problem, is to provide a container for waste syringe needles that can safely separate a needle unit from a used pen syringe and house the separated needle unit immediately after the separation without hand contact with the needle unit.

### SOLUTION TO PROBLEM

To achieve the above object, a container for waste syringe needles according to the present disclosure includes a container body configured to house a needle unit of a disposable pen syringe; a lid disposed on the top of the container body, the lid including an insertion opening configured to receive the needle unit, the insertion opening including a rotation restrictor configured to prevent the needle unit from rotating; and an openable closer attached to the lid, the closer including a pusher on the lower surface, the closer being configured to cover the lid, wherein when the needle unit is inserted into the insertion opening and the syringe is rotated against the needle unit while the needle unit is fixed against rotation, the needle unit is separated from the syringe while remaining in the insertion opening, and when the closer is closed, the pusher pushes the needle unit downward and drops the needle unit from the insertion opening into the container body.

### ADVANTAGEOUS EFFECTS OF INVENTION

A container for waste syringe needles according to the present disclosure can safely separate needle units from used pen syringes and safely house the separated needle units alone, without use of needle cases.

### BRIEF DESCRIPTION OF DRAWINGS

FIG. 1 is a perspective view of a container for waste syringe needles with an opened closer;

FIG. 2 is an exploded perspective view of a pen syringe;

FIG. 3 is a perspective view of a container for waste syringe needles during separation of a needle unit from a pen syringe;

FIG. 4 is a perspective view of a container for waste syringe needles after separation of a needle unit from a pen syringe;

FIG. 5 is a perspective view of a container for waste syringe needles during closure of a closer;

FIG. 6 is a perspective view of a capped syringe;

FIG. 7 is a perspective view of a container for waste syringe needles during separation of a needle unit from a capped syringe;

FIG. 8 is a perspective view of a locking syringe;

FIG. 9 is a perspective view of a container for waste syringe needles during separation of a needle unit from a locking syringe;

FIG. 10 is a perspective view of a container for waste syringe needles during disposal of a butterfly needle with a tube; and

FIG. 11 is a perspective view of a container for waste syringe needles with a closed closer.

### DESCRIPTION OF EMBODIMENTS

The present invention will now be described in detail based on embodiments illustrated in the drawings.

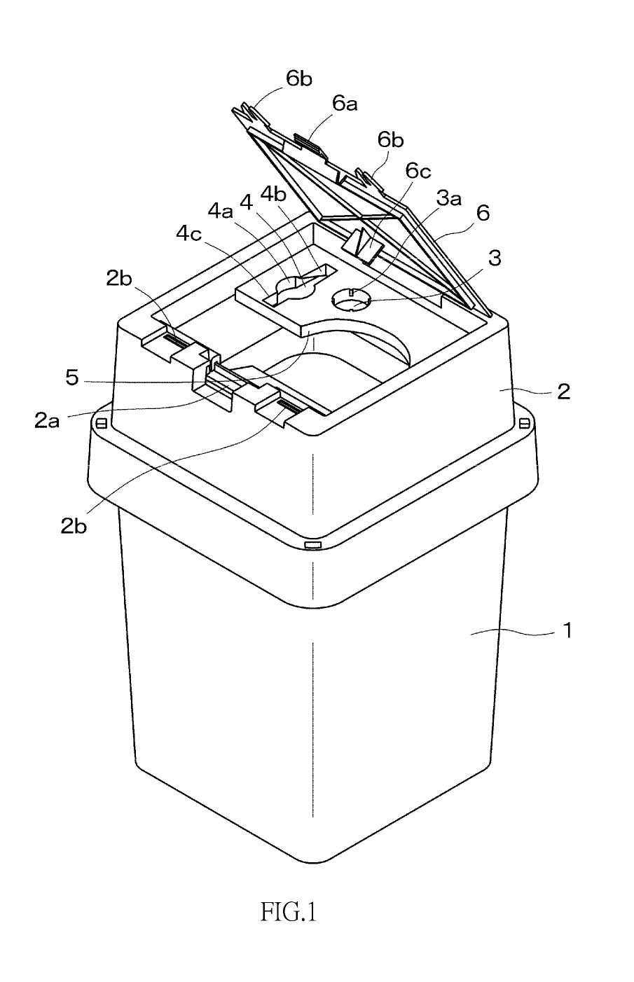

FIG. 1 is a perspective view of a container for waste syringe needles according to the present embodiment. For example, a container body 1 made of synthetic resin, such as polypropylene, is provided with a plastic lid 2 tightly fitting to the top. The lid 2 may be fixed to the container body 1 so as not to be separated therefrom. The lid 2 includes an insertion opening 3 at its top plate. The insertion opening 3 receives a needle unit and separates the needle unit from a pen syringe for self-injection of solution of a drug such as insulin or growth hormone. The insertion opening 3 has one or more vertical linear protrusions 3a thereinside as a rotation restrictor.

The top plate of the lid 2 further includes an opening 4 that separates a needle unit from a capped or locking syringe for general purposes such as blood collection and vaccination. The opening 4 consists of a central circular opening 4a and adjoining rectangular openings 4b and 4c each defined by three inner walls. The opposing inner walls of the rectangular opening 4b have bottoms 4d inclined downwardly toward the third inner wall, while the inner walls of the rectangular opening 4c have a constant vertical length. The lid 2 further includes a disposal opening 5 large enough to accept a butterfly needle with a tube for drip infusion, for example.

The lid 2 has an openable closer 6 for covering the lid 2 attached at a side of the lid 2 with a hinge. The closer 6 includes a locking claw 6a to engage with a groove 2a provided on the lid 2 and two bendable locking claws 6b to individually fit into grooves 2b on the lid 2 at the distal end from the hinge. The lower surface of the closer 6 has a projecting pusher 6c with a cross-shaped end so that the pusher 6c is disposed in the insertion opening 3 of the lid 2 after the closer 6 is closed.

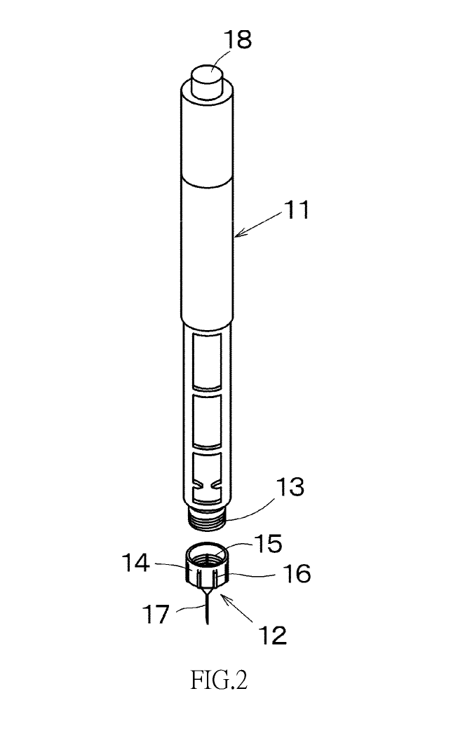

FIG. 2 is an exploded perspective view of a pen syringe which includes a syringe 11 and a needle unit 12. The syringe 11 has an external thread 13 around the lower end. The needle unit 12 includes a cylindrical plastic base 14 having an internal thread groove 15 mating with the external thread 13 and having longitudinal linear protrusions 16 around the outer periphery of the base 14. The base 14 is provided with a syringe needle 17 vertically extending therethrough.

The external thread 13 of the syringe 11 is screwed into the thread groove 15 of the needle unit 12, so that the base 14 and the syringe 11 are coupled to serve as a syringe. In specific, with the base end of the syringe needle 17 extending into the syringe 11, pushing an injection button 18 of the syringe 11 causes solution of a drug such as insulin in the syringe 11 to exit through the syringe needle 17.

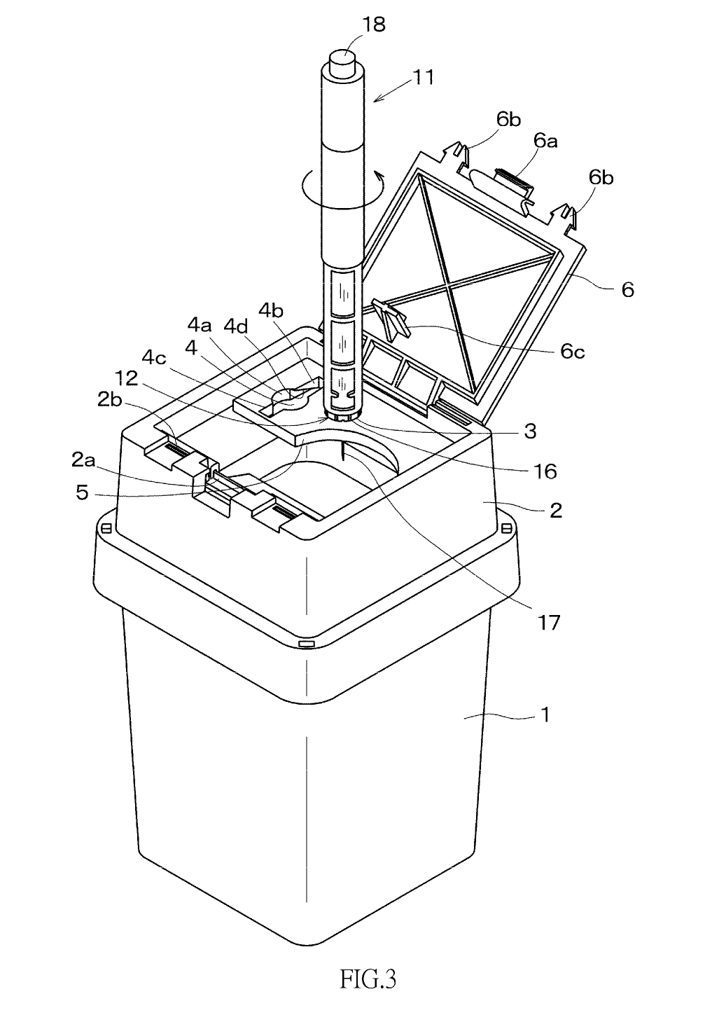

FIG. 3 is a perspective view of a container for waste syringe needles during separation of a used needle unit 12 from a pen syringe. The insertion opening 3 has an inner diameter slightly smaller than the outer diameter of the base 14 of the needle unit 12. When a user opens the closer 6 and inserts the needle unit 12 into the insertion opening 3 while holding the syringe 11, the vertical linear protrusions 3a inside the insertion opening 3 engage with the linear protrusions 16 of the needle unit 12 in the rotational direction.

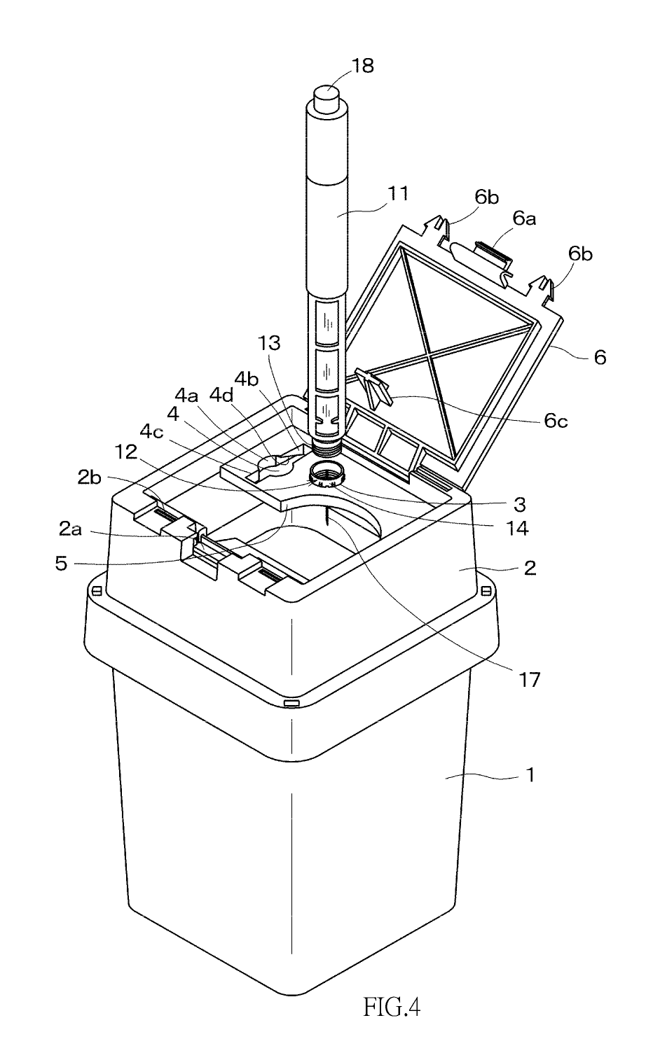

After insertion of the needle unit 12 into the insertion opening 3, rotation of the syringe 11 along the arrow unscrews the needle unit 12 from the syringe 11 without further rotation of the needle unit 12 and can therefore separate the needle unit 12 from the syringe 11 as illustrated in FIG. 4. In brief, the syringe 11 can be separated from the needle unit 12 remaining in the insertion opening 3.

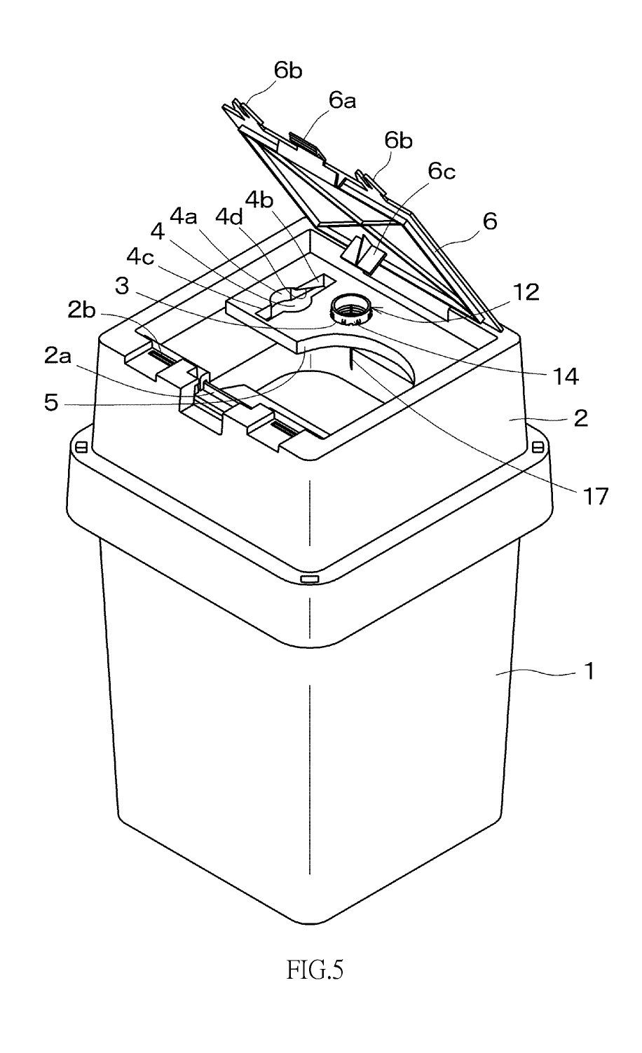

When the closer 6 is halfway closed while the separated needle unit 12 remains in the insertion opening 3 as illustrated in FIG. 5, the pusher 6c comes in contact with the top of the needle unit 12. Further closure of the closer 6 allows the needle unit 12 to be pushed downward by the pusher 6c and to fall away from the insertion opening 3 into the container body 1. This enables the needle unit 12 to be safely housed in the container body 1 without hand contact with the needle unit 12.

After the closure of the closer 6 and the housing of the needle unit 12, the locking claw 6a of the closer 6 engages with the groove 2a of the lid 2, thereby maintaining the closer 6 to be closed. The closer 6 can be opened by disengagement of the locking claw 6a.

While the needle unit 12 is discarded after every injection, the syringe 11 can be repeatedly used with a new needle unit 12 for the same patient.

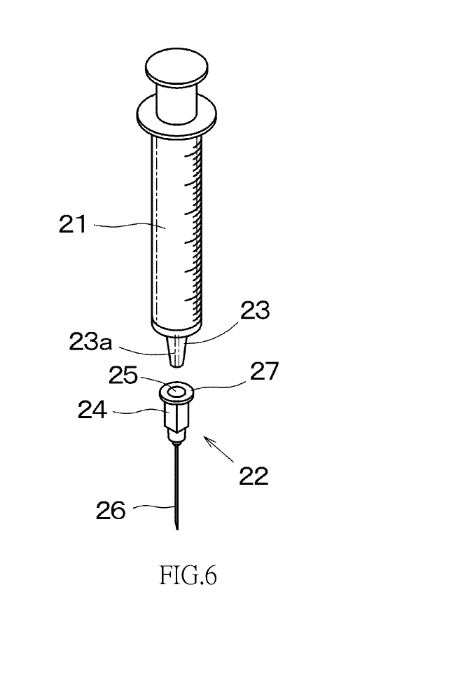

FIG. 6 is an exploded perspective view of a general-purpose capped syringe, which includes a syringe 21 and a needle unit 22. The syringe 21 is provided with a taper 23 at the tip. The taper 23 has a through hole 23a for drug solution flowing therethrough. The needle unit 22 includes a plastic base 24 having a tapered hole 25 that can fit the taper 23 of the syringe 21 at the center. The bottom of the base 24 is fixed to a syringe needle 26 in communication with the tapered hole 25. The base 24 has a square cross-section and includes a flange 27 at the top. The taper 23 is fit to the tapered hole 25, so that the syringe 21 and the needle unit 22 are coupled to serve as a syringe.

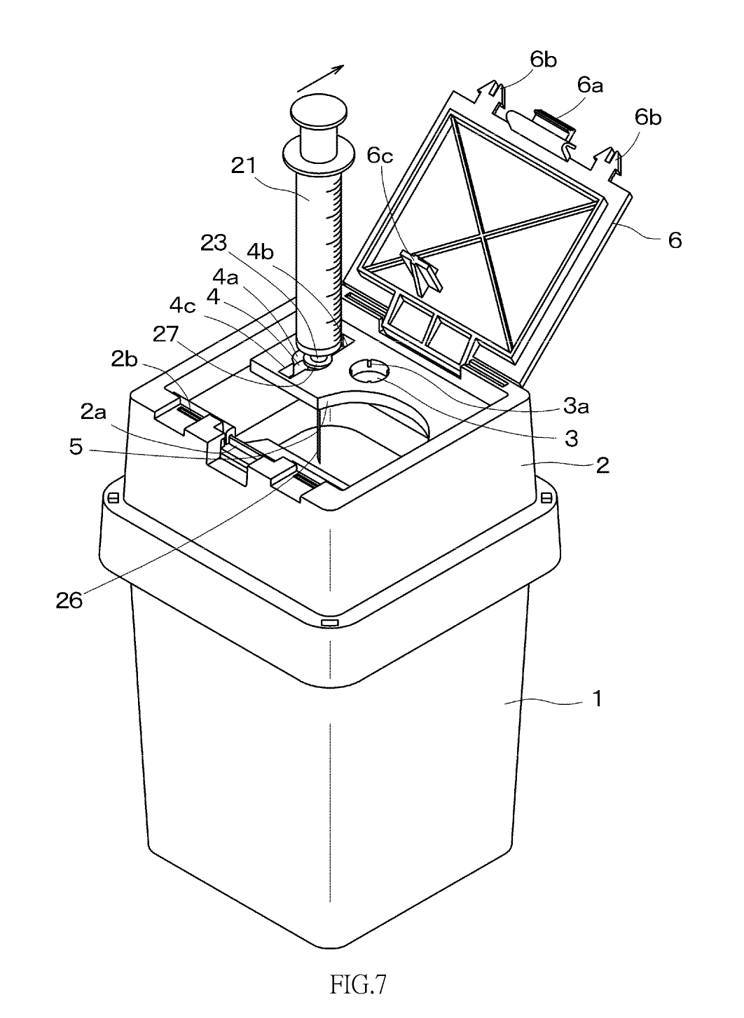

As illustrated in FIG. 7, a user slides the syringe 21 along the arrow while bringing the upper surface of the flange 27 of the needle unit 22 into contact with the bottoms 4d, which form the back of the rectangular opening 4b of the opening 4 in the lid 2 and incline downward toward the distal end from the circular opening 4a as described above. The needle unit 22 is accordingly extracted from the syringe 21 by the effect of the bottoms 4d and falls into the container body 1.

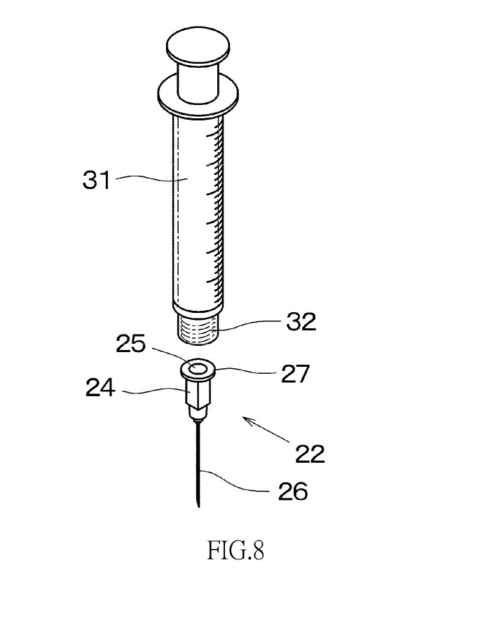

FIG. 8 is an exploded perspective view of a general-purpose locking syringe. The locking syringe and the capped syringe can use a common needle unit 22, and the needle unit 22 will not be described in detail below.

In the locking syringe, a thread groove 32 is provided inside the tip-side cylindrical portion of a syringe 31. The flange 27, which constitutes the thread of the needle unit 22, is screwed into the thread groove 32, so that the syringe 31 and the needle unit 22 are coupled to serve as a syringe.

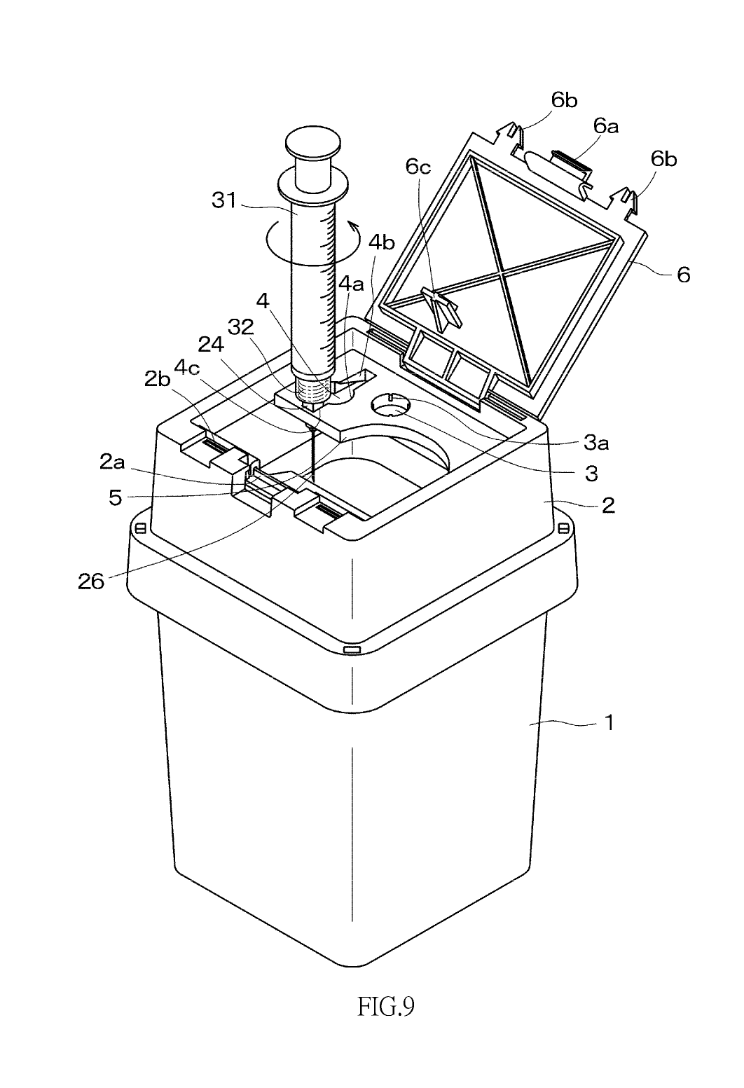

Upon separation of the needle unit 22 from a used locking syringe, a user fits the square cross-sectional base 24 of the needle unit 22 into the rectangular opening 4c as illustrated in FIG. 9. When the syringe 31 is rotated along the arrow, the needle unit 22 is prevented from rotating by the rectangular opening 4c and is unscrewed from the syringe 31. When the tip of the syringe 31 is moved to the circular opening 4a, the separated needle unit 22 falls into the container body 1.

Although the present embodiment employs the rectangular opening 4c and the square cross-sectional base 24 for separation of a needle unit 22, these components may have any other shape.

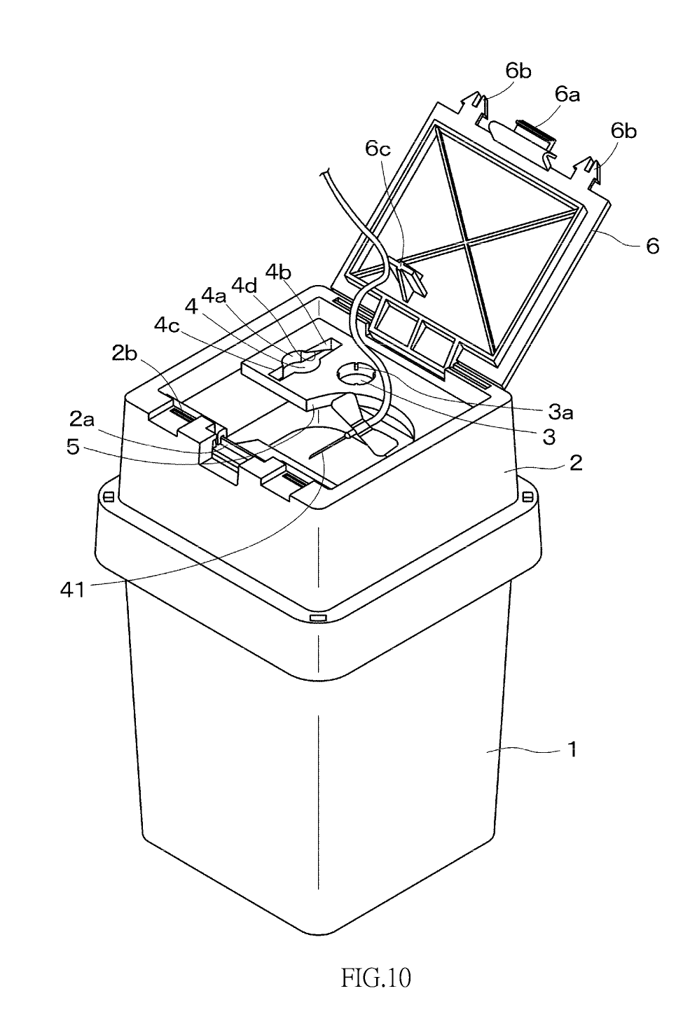

As illustrated in FIG. 10, the disposal opening 5 can accept a butterfly needle 41 with a tube, for example.

It is preferred that the closer 6 be kept closed after the separation of a needle unit 22 from a capped or locking syringe. Even if the container for waste syringe needles falls down, the closure of the closer 6 prevents the contents such as needle units 12 and 22 and butterfly needles 41 from spilling out through the disposal opening 5.

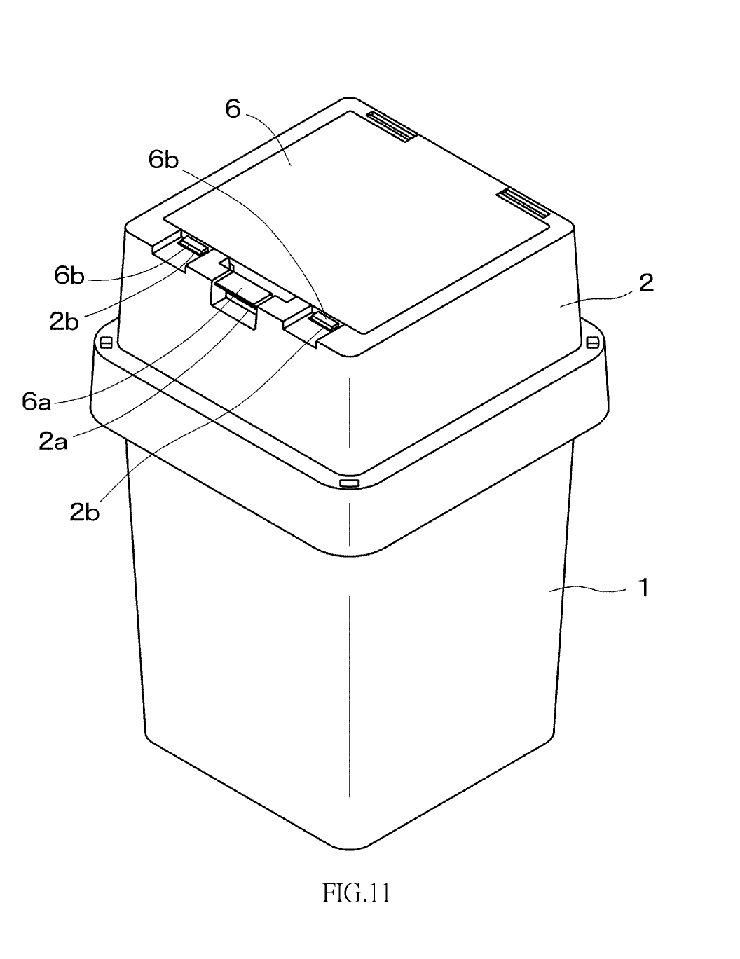

As illustrated in FIG. 11, the bent two locking claws 6b at the end of the closer 6 tightly fit into the grooves 2b on the top of the lid 2, and the groove 6a of the closer 6 engages with the groove 2a, so that the closer 6 is locked. This enables the container for waste syringe needles to be safely discarded as it is when the container is filled up with used needle units 12 and 22, butterfly needles 41, and other components.

The container body 1 may include a detachable inclined plate, which is not illustrated in the present embodiment, below the disposal opening 5 as required, in order to prevent a needle unit 12 or 22 falling in the container body 1 from bouncing on the bottom of the container body 1 and flying out through the disposal opening 5.

Although the container for waste syringe needles according to the present embodiment includes the insertion opening 3 and the opening 4 and can therefore safely separate and house a used syringe needle for any of pen syringes and general-purpose capped and locking syringes, the container may include only the insertion opening 3 and may achieve separation and housing for only the needle units 12 of the pen syringes.

What is claimed is:

1. A container for waste syringe needles, comprising:
a container body configured to house a needle unit of a disposable pen syringe;
a lid disposed on the top of the container body, the lid comprising an insertion opening configured to receive the needle unit, the insertion opening comprising a rotation restrictor configured to prevent the needle unit from rotating; and
an openable closer attached to the lid, the closer comprising a pusher on the lower surface, the closer being configured to cover the lid, wherein
when the needle unit is inserted into the insertion opening and the syringe is rotated against the needle unit while the needle unit is fixed against rotation, the needle unit is separated from the syringe while remaining in the insertion opening, and
when the closer is closed, the pusher pushes the needle unit downward and drops the needle unit from the insertion opening into the container body.

2. The container for waste syringe needles according to claim 1, wherein the rotation restrictor comprises one or more vertical linear protrusions inside the insertion opening and engages with linear protrusions provided around the needle unit in a rotational direction.

3. The container for waste syringe needles according to claim 1, wherein the closer is coupled to the lid through a hinge.

4. The container for waste syringe needles according to claim 1, wherein
the lid further comprises a lock groove,
the closer further comprises a lock claw, and
upon disposal of the container, the lock claw tightly fits into the lock groove such that the closer is locked.

5. The container for waste syringe needles according to claim 2, wherein the closer is coupled to the lid through a hinge.

6. The container for waste syringe needles according to claim 2, wherein
the lid further comprises a lock groove,
the closer further comprises a lock claw, and
upon disposal of the container, the lock claw tightly fits into the lock groove such that the closer is locked.
